(19) United States
(12) Patent Application Publication
(10) Pub. No.: US 2020/0254068 A1
(43) Pub. Date: Aug. 13, 2020

(54) METHODS FOR TREATING TRAUMATIC BRAIN INJURY

(71) Applicant: LOMA LINDA UNIVERSITY, Loma Linda, CA (US)

(72) Inventors: Stephen ASHWAL, Loma Linda, CA (US); Barbara HOLSHOUSER, Loma Linda, CA (US)

(21) Appl. No.: 16/760,487

(22) PCT Filed: Oct. 30, 2018
(86) PCT No.: PCT/US2018/058276

§ 371 Date: Apr. 30, 2020

Related U.S. Application Data
(60) Provisional application No. 62/746,381, filed on Oct. 16, 2018.
(60) Provisional application No. 62/579,832, filed on Oct. 31, 2017.

(51) Int. Cl. A61K 38/35

(52) U.S. Cl. CPC A61K 38/35

(57) ABSTRACT

In one aspect, the disclosure provides methods of treating traumatic brain injury (TBI) in a subject by administering to the subject an adrenocorticotropic hormone (ACTH). In some embodiments, the subject is administered a synthetic derivative of ACTH, e.g., cosyntropin.

### CROSS-REFERENCE TO RELATED APPLICATIONS

This application claims priority to U.S. Provisional Application No. 62/579,832, filed Oct. 31, 2017, and to U.S. Provisional Application No. 62/746,381, filed Oct. 16, 2018, the entire contents of each of which are incorporated by reference herein.

### BACKGROUND

Every year, an estimated 42 million people worldwide suffer a traumatic brain injury (TBI). In the United States alone, TBI is a major cause of death and disability. In 2013 in the United States, TBI was a diagnosis in more than 282,000 hospitalizations and 2.5 million emergency department visits. Additionally, TBI contributed to the deaths of nearly 50,000 people (US Department of Health and Human Services, Centers for Disease Control and Prevention). It is estimated that approximately 5.3 million people in the United States and nearly 7.7 million people in Europe are living with TBI-related disability (Rubiano et al., Nature 527:S193, 2015). Chronic post-concussion symptoms (PCS) and chronic traumatic encephalopathy (CTE) are increasingly being recognized in active and retired National Football League (NFL) players. Although their underlying causes are under investigation, it is likely that there are multiple etiologies.

Currently, treatment for TBI and chronic mild traumatic brain injury (mTBI) remains indirect and essentially targets symptomatic alleviation. The current standard of care following TBI is limited in both scope and effectiveness resulting in the need for novel therapies (Abou El Fadl and O'Phelan, 2018). Moreover, at least in part because of an incomplete understanding of the mechanisms responsible for TBI and mTBI, there currently are no safe and effective treatments to ameliorate the sensorimotor and neuropsychological consequences of TBI. Neuroinflammation has drawn much interest as a potential therapeutic target in TBI patients as increasing evidence has shown that it contributes to injury progression in chronic traumatic encephalopathy, demyelinating diseases, and a variety of other well-known neurological disorders (McKee and Lukens, Front Immunol 7:556, 2016). As a result, inflammation and glucose metabolism are attractive therapeutic targets for the treatment of TBI. Melanocortin receptor 4 (MCR4) is one of five melanocortin receptors and is expressed in the central nervous system, including hypothalamus, cortex, and hippocampus (Kishi et al., 2003). Agonists for MCR4 (e.g., adrenocorticotropic hormone (ACTH), α-MSH) have been previously shown to be neuroprotective and anti-inflammatory (Catania, 2004, 2008; Giuliani et al., 2014; Lisak et al., 2015; Morgan et al., 2015).

In models of brain injury and inflammation, MCR4 signaling demonstrated the ability to inhibit NFkB translocation into the nucleus, decrease inflammatory markers, and increase AMP-activated protein kinase (AMPK) activity (Catania, 2004; Chen et al., 2018; Ichiyama et al., 1999). Following TBI, microglia exhibit an activated state composed of both M1 and M2 phenotypes, classically described as pro- and anti-inflammatory, respectively (Donat et al., 2017). While NFkB increases M1 phenotype, AMPK activity has been shown to enhance microglia M2 polarization (Wang et al., 2018). AMPK is also a master metabolic regulator that senses states of low energy and enhances energy production (Hardie et al., 2012; Rabinovitch et al., 2017). Pharmacological enhancement of AMPK activity following experimental TBI is associated with improved cognitive outcomes and increased glucose metabolism (Hill et al., 2016). There is limited research investigating neuroprotective effects of melanocortin receptor signaling following TBI.

### SUMMARY

In one aspect, the disclosure features a method of treating traumatic brain injury, comprising administering to a subject having a TBI a therapeutically effective amount of an adrenocorticotropic hormone (ACTH).

In some embodiments of this aspect, the subject has neuroinflammation and/or Tau deposition in one or more brain regions.

In some embodiments of this aspect, the traumatic brain injury is caused by a fall, an assault, a motor vehicle accident, a sport or recreational injury, shaken baby syndrome, a gunshot wound, a combat injury, or an electric shock.

In another aspect, the disclosure features a method of reducing neuroinflammation and/or Tau deposition in one or more brain regions in a subject in need thereof, comprising administering to the subject a therapeutically effective amount of an ACTH.

In some embodiments of the methods described herein, the ACTH is a full-length ACTH polypeptide. In some embodiments, the ACTH is a synthetic derivative of a full-length ACTH polypeptide. In particular embodiments, the ACTH is cosyntropin.

In some embodiments of the methods described herein, the neuroinflammation and/or Tau deposition may be identified by magnetic resonance imaging (MRI) and/or positron emission tomography (PET) imaging.

In some embodiments of the methods described herein, the subject has one or more neurological symptoms selected from the group consisting of memory loss, depression, mood swings, balance problems, anger, aggression, anxiety, substance abuse, obsessive compulsive disorder, and muted emotions.

In some embodiments, the treatment with the ACTH increases brain glucose metabolism. In more embodiments, the treatment with the ACTH decreases microglia activation. In other embodiments, the treatment with the ACTH may alter microglia cell count (i.e., may increase microglia cell count).

In some embodiments, the treatment with the ACTH increases microglia cell area. In other embodiments, the treatment with the ACTH decreases microglia cell density. In further embodiments, the treatment with the ACTH increases microglia cell perimeter.

### BRIEF DESCRIPTION OF THE DRAWINGS

FIG. 1 shows a schematic outline of a neuroinflammation clinical study design. In this study, former NFL players with chronic (≥6 months) CNS symptoms and age-matched controls are evaluated. SCAT3=Sports Concussion Assessment Tool; BESS=Balance Error Scoring System; GDS=Geriatric Depression Scale; MoCA=Montreal Cognitive Assessment Tool; MRI=magnetic resonance imaging with perfusion, diffusion tensor; MRS=MR Spectroscopy; neuroinflammation PET=Neuroinflammation PET Imaging with ¹¹C-DPA-713; Tau PET=Tau PET imaging with ¹⁸F-MK6240; SRMT=Standard Rehabilitation Medical Treatment; NP=Neuropsychological assessment.

FIG. 2 shows an exemplary multiparametric MRI/MRS post-processing pipeline used to generate quantitative regional measures of brain diffusivity and metabolite ratios (modified from Ghosh et al., Dev Neurosci 39:413, 2016). AD, axial diffusivity; ADC, apparent diffusion coefficient; CSF, cerebral spinal fluid; DTI, diffusion tensor imaging; FA, fractional anisotropy; GM, gray matter; MRSI, MR spectroscopic imaging; RD, radial diffusivity; TBSS, tract-based spatial statistics; and WM, white matter.

FIGS. 3A-3G show the advanced magnetic resonance imaging (MRI) analysis performed on the brain injury in a 12-year-old boy (Ashwal et al., J Child Neurol. 29:1704, 20104).

FIG. 4 shows IBA-1 staining for microglia with 4× (upper row), 40× (middle) and 100× (lower) images of CCI injured and sham (craniotomy) rat brains treated with cosyntropin (120 units/kg/day×7 days) or saline.

FIG. 5 shows an increased number of perilesional microglia following injury but no difference with cosyntropin treatment vs saline. Error bars indicate standard deviation.

FIG. 6 shows a schematic image depicting the process of image analysis with FracLac for ImageJ.

FIGS. 7A-7D show that cosyntropin reduces CCI-induced morphological changes in microglia. (A) Microglia cell area. (B) Cell perimeter. (C) Density of microglia. (D) Microglia cell circularity.

FIG. 8 shows a proposed mechanism for protective effect of cosyntropin following TBI.

FIG. 9 shows an experimental design for assessing the effect of cosyntropin following the induction of experimental TBI.

### DETAILED DESCRIPTION OF THE EMBODIMENTS

### I. Introduction

Many current and former NFL players have neurological symptoms due to repeated mild head injury and concussions. Symptoms include memory loss, depression, mood swings, balance problems, and behavioral changes. There is evidence that brain inflammation occurs after repeated injuries and contributes to a player's symptoms and/or to the development of CTE. TBI results in prolonged secondary sequelae, including neuroinflammation and suppressed glucose metabolism contributing to disturbed cortical and hippocampal inflammation and increased risk of neurodegeneration (Faden and Loane, 2015; Simon et al., 2017; Xiong et al., 2018). Neuroinflammation has drawn interest as a potential therapeutic target in TBI patients as increasing evidence has shown that it contributes to injury progression in chronic traumatic encephalopathy, demyelinating diseases, and a variety of other well-known neurological disorders (McKee and Lukens, Front Immunol 7:556, 2016). Neuroinflammation may mediate several different pathways that are activated after TBI including: (1) activation of endogenous CNS immunocompetent cells such as microglia; (2) altering blood brain barrier structure/function; and (3) initiating the infiltration of circulating activated immune cells into the central nervous system (CNS) (Albrecht et al., ACS Chem Neurosci. 7:470, 2016).

Melanocortin receptor 4 (MCR4) is one of five melanocortin receptors and is expressed in the central nervous system, including hypothalamus, cortex, and hippocampus (Kishi et al., 2003). Agonists for MCR4 have been previously shown to be neuroprotective and anti-inflammatory (Catania, 2004, 2008; Giuliani et al., 2014; Lisak et al., 2015; Morgan et al., 2015). In models of brain injury and inflammation, MCR4 signaling demonstrated the ability to inhibit NFkB translocation into the nucleus, decrease inflammatory markers, and increase AMP-activated protein kinase (AMPK) activity (Catania, 2004; Chen et al., 2018; Ichiyama et al., 1999). Following TBI, microglia exhibit an activated state composed of both M1 and M2 phenotypes, classically described as pro- and anti-inflammatory, respectively (Donat et al., 2017). While NFkB increases M1 phenotype, AMPK activity has been shown to enhance microglia M2 polarization (Wang et al., 2018). AMPK is also a master metabolic regulator that senses states of low energy and enhances energy production (Hardie et al., 2012; Rabinovitch et al., 2017). Pharmacological enhancement of AMPK activity following experimental TBI is associated with improved cognitive outcomes and increased glucose metabolism (Hill et al., 2016). However, there is limited research investigating neuroprotective effects of melanocortin receptor signaling following TBI.

In the past, corticosteroids have been studied in clinical TBI trials but were found to either be unsuccessful (progesterone) or to increase the mortality rate (dexamethasone) (see, Wright et al., New Engl J Med. 371:2457, 2014). In these studies, the corticosteroids were given acutely and primarily with the goal of reducing cerebral edema. As described herein, adrenocorticotropic hormone (ACTH or corticotropin) has emerged as an alternative for patients who do not respond to or do not tolerate corticosteroids and in whom administration over the course of injury to reduce neuroinflammation may be a more biologically plausible goal. As described in the Examples section below, a synthetic ACTH analog, cosyntropin, has been found to reduce microglia activation in a rodent model of TBI. Thus, the anti-inflammatory roles and mechanisms of ACTH may limit the neuroinflammatory impairments associated with TBI and result in improved motor and neurocognitive outcomes in subjects.

### II. Definitions

The terminology used herein is for the purpose of describing particular embodiments only, and is not intended to be limiting, because the scope of the present invention will be limited only by the appended claims. Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings unless a contrary intention is apparent. In some cases, terms with commonly understood meanings are defined herein for clarity and/or for ready reference, and the inclusion of such definitions herein should not be construed as representing a substantial difference over the definition of the term as generally understood in the art.

All numerical designations, e.g., pH, temperature, time, concentration, and molecular weight, including ranges, are approximations which are varied (+) or (−) by increments of 0.1 or 1.0, as appropriate. It is to be understood, although not always explicitly stated that all numerical designations are preceded by the term “about.”

The singular forms “a,” “an,” and “the” include plural referents unless the context clearly dictates otherwise. Thus, for example, reference to “a compound” includes a plurality of compounds.

The term “comprising” is intended to mean that the compounds, compositions and methods include the recited elements, but not excluding others. “Consisting essentially of” when used to define compounds, compositions and methods, shall mean excluding other elements that would materially affect the basic and novel characteristics of the claimed invention. “Consisting of” shall mean excluding any element, step, or ingredient not specified in the claim. Embodiments defined by each of these transition terms are within the scope of this invention.

As used herein, the term “traumatic brain injury” or “TBI” refers to a direct or indirect damage to the brain or head, generally from a physical force. A TBI may be confined to one area of the brain or head or involve more than one area of the brain or head. Some internal symptoms or findings of TBI include, but are not limited to, neuroinflammation, hypotension, hypoxia, edema, abnormalities in glucose utilization, changes in cellular metabolism, changes in membrane fluidity, changes in synaptic function, and changes in structural integrity of the brain. Some external symptoms of TBI include, but are not limited to, memory loss, depression, mood swings, balance problems, and behavioral changes (e.g., display of anger, aggression, anxiety, substance abuse, obsessive compulsive disorder, and muted emotions).

As used herein, the term “adrenocorticotropic hormone” or “ACTH” refers to a peptide hormone produced and secreted by the anterior pituitary gland that stimulates the adrenal cortex to secrete glucocorticoids such as cortisol, or a derivative thereof. ACTH is also known in the art as “corticotropin.” In some embodiments, an ACTH is a full-length ACTH polypeptide. In some embodiments, an ACTH is a truncated form of ACTH that retains the activity of full-length ACTH. An ACTH or a derivative thereof may be natural (e.g., a naturally occurring from) or synthetic (e.g., a non-naturally occurring form)

The terms “protein” and “polypeptide” are used interchangeably herein and refer to a polymer of amino acid residues. As used herein, the terms encompass amino acid chains of any length, including full-length proteins and truncated proteins.

As used herein, the term “microglia cell area” refers to the two-dimensional area occupied by an microglia cell. In some embodiments, microglia cell area is calculated or counted by the number of pixels from a microscopic image of the microglia cell.

As used herein, the term “microglia cell density” refers to the microglia cell area per convex hull area. A convex hull refers to the smallest convex polygon having all interior angles smaller than 180 degrees that contains the whole cell shape (see, e.g., Fernández-Arjona, et al., Front Cell Neurosci. 11:235, 2017).

As used herein, the term “microglia cell perimeter” refers to the outline of a microglia cell. In some embodiments, microglia cell perimeter is calculated or counted by the number of pixels from a microscopic image of the microglia cell.

As used herein, a “subject” is a mammal, in some embodiments, a human. Mammals can also include, but are not limited to, farm animals (e.g., cows, pigs, horses, chickens, etc.), sport animals, pets, primates, horses, dogs, cats, and rodents (e.g., mice and rats).

As used herein, the terms “treatment,” “treating,” and “treat” refer to any indicia of success in the treatment or amelioration of an injury, disease, or condition, including any objective or subjective parameter such as abatement; remission; diminishing of symptoms or making the injury, disease, or condition more tolerable to the subject; slowing in the rate of degeneration or decline; making the final point of degeneration less debilitating; and/or improving a subject's physical or mental well-being.

As used herein, a “therapeutic amount” or a “therapeutically effective amount” of an agent (e.g., ACTH or a derivative thereof) is an amount of the agent that prevents, alleviates, abates, or reduces the severity of symptoms of a disease (e.g., TBI) in a subject. For example, for the given parameter, a therapeutically effective amount will show an increase or decrease of therapeutic effect of at least 5%, 10%, 15%, 20%, 25%, 40%, 50%, 60%, 75%, 80%, 90%, or at least 100%. Therapeutic efficacy can also be expressed as “fold” increase or decrease. For example, a therapeutically effective amount can have at least a 1.2-fold, 1.5-fold, 2-fold, 5-fold, or more effect over a control.

The terms “administer,” “administered,” or “administering,” as used herein, refer to introducing an agent (e.g., ACTH or a derivative thereof) into a subject or patient, such as a human. As used herein, the terms encompass both direct administration, (e.g., self-administration or administration to a patient by a medical professional) and indirect administration (e.g., the act of prescribing a compound or composition to a subject).

As used herein, the term “pharmaceutical composition” refers to a composition suitable for administration to a subject. In general, a pharmaceutical composition is sterile, and preferably free of contaminants that are capable of eliciting an undesirable response with the subject. Pharmaceutical compositions can be designed for administration to subjects in need thereof via a number of different routes of administration, including oral, intravenous, buccal, rectal, parenteral, intraperitoneal, intradermal, intranasal, intraventricular, intratracheal, intramuscular, subcutaneous, inhalational, transdermal, and the like.

### III. Adrenocorticotropic Hormone

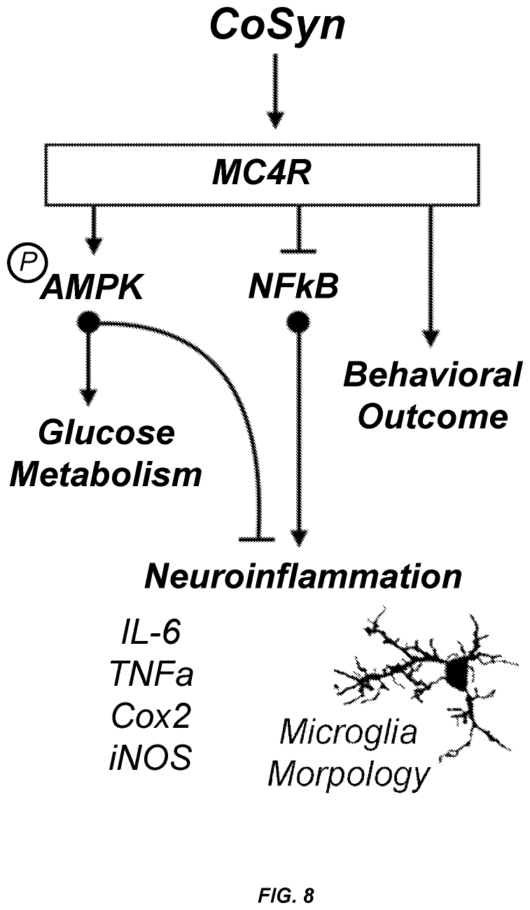

In one aspect, the disclosure relates to the use of an adrenocorticotropic hormone (ACTH) or a derivative thereof for treating traumatic brain injury in a subject and/or for reducing neuroinflammation and/or Tau deposition in one or more brain regions in a subject. ACTH is a peptide hormone produced and secreted by the anterior pituitary gland that stimulates the adrenal cortex to secrete glucocorticoids such as cortisol. As described herein, without being bound by any theory, it is hypothesized that an ACTH or a derivative thereof (e.g., cosyntropin), through activation of one or more melanocortin receptors (e.g., melanocortin receptor 4 (MCR4)), may reduce neuroinflammation, for example by reducing NFKB translocation and increase AMP-activated protein kinase (AMPK) activity, which subsequently leads to an anti-inflammatory response (see, e.g., FIG. 8).

In some embodiments, an ACTH is a natural ACTH hormone. In some embodiments, the ACTH is the human full-length ACTH polypeptide having the amino acid sequence SYSMEHFRWGKPVGKKRRPVKVYPNGAEDESAEAFPLEF (SEQ ID NO:1). In some embodiments, a natural ACTH hormone (e.g., a polypeptide having the sequence of SEQ ID NO:1) may refer to a naturally occurring ACTH hormone, such as an ACTH hormone extracted and purified from mammalian pituitary glands. In other embodiments, a natural ACTH hormone may also refer to a hormone that occurs in nature (e.g., a polypeptide having the sequence of SEQ ID NO:1) but that is synthetically made via, for example, peptide synthesis.

In some embodiments, an ACTH is a synthetic ACTH hormone (i.e., a non-naturally occurring form, e.g., a derivative of a full-length ACTH polypeptide). In some embodiments, a synthetic ACTH hormone is a truncated form of an ACTH polypeptide (e.g., human ACTH polypeptide) that substantially retains the activity of the full-length ACTH from which it is derived. For example, in some embodiments, the ACTH is a truncated form of full-length human ACTH, e.g., a polypeptide having the amino acid sequence SYSMEHFRWGKPVGKKRRPVKVYP (SEQ ID NO:2) (ACTH₁₋₂₄). Examples of synthetic ACTH hormones include, but are not limited to, cosyntropin (also known in the art as tetracosactide and tetracosactrin), and the acetate ester form of cosyntropin, commonly referred to as tetracosactide acetate or tetracosactrin acetate.

In some embodiments, cosyntropin has the sequence of SEQ ID NO:2 and substantially retains the function of the full-length ACTH (e.g., the polypeptide having the sequence of SEQ ID NO:1).

An ACTH as described herein can be synthesized chemically using conventional peptide synthesis or other protocols well known in the art, by recombinant expression, or can be obtained from natural sources.

Peptides may be synthesized by solid-phase peptide synthesis methods using procedures similar to those described by Merrifield et al., J. Am. Chem. Soc., 85:2149-2156 (1963); Barany and Merrifield, Solid-Phase Peptide Synthesis, in The Peptides: Analysis, Synthesis, Biology Gross and Meienhofer (eds.), Academic Press, N.Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal and to a solid support, i.e., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile. Details of appropriate chemistries, resins, protecting groups, protected amino acids and reagents are described in the art. See, e.g., Atherton et al., Solid Phase Peptide Synthesis: A Practical Approach, IRL Press (1989), and Bodanszky, Peptide Chemistry, A Practical Textbook, 2nd Ed., Springer-Verlag (1993)).

Materials suitable for use as the solid support are well known to those of skill in the art and include, but are not limited to, the following: halomethyl resins, such as chloromethyl resin or bromomethyl resin; hydroxymethyl resins; phenol resins, such as 4-(α-[2,4-dimethoxyphenyl]-Fmoc-aminomethyl)phenoxy resin; tert-alkyloxycarbonyl-hydrazidated resins, and the like. Such resins are commercially available and their methods of preparation are known by those of ordinary skill in the art.

ACTH polypeptides can also be produced by recombinant expression. Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994). In some embodiments, ACTH or a derivative thereof is produced by recombinant expression in bacteria, such as E. coli, yeast cells, insect cells, or mammalian cells.

### VI. Therapeutic Methods

In one aspect, the disclosure provides methods of treating traumatic brain injury (TBI) by administering to a subject having a TBI a therapeutically effective amount of an adrenocorticotropic hormone (ACTH). A TBI may be caused by any external force, such as a fall, an assault, a motor vehicle accident, a sport or recreational injury, shaken baby syndrome, a gunshot wound, a combat injury, or an electric shock. In some embodiments, a subject with TBI may have one or more neurological symptoms, such as memory loss, depression, mood swings, balance problems, and behavioral changes (e.g., display of anger, aggression, anxiety, substance abuse, obsessive compulsive disorder, and muted emotions).

In another aspect, the disclosure provides methods of reducing neuroinflammation and/or Tau deposition in one or more brain regions in a subject by administering to the subject a therapeutically effective amount of an adrenocorticotropic hormone (ACTH). In some embodiments, the subject has a TBI. In some embodiments, a subject has mild traumatic brain injury (mTBI). In some embodiments, the subject has one or more neurological symptoms, such as memory loss, depression, mood swings, balance problems, and other behavioral changes (e.g., display of anger, aggression, anxiety, substance abuse, obsessive compulsive disorder, and muted emotions).

Identifying Subjects

In some embodiments, a subject to be treated according to the methods disclosed herein has TBI. The severity of a TBI can be estimated using one or more tests known in the art, such as but not limited to, Glasgow Coma Scale (GCS) score, measurements for level of TBI (e.g., ranking a person's level of consciousness, memory loss, and GCS), speech and language tests, cognition and neuropsychological tests, and imaging tests. See, www.nichd.nih.gov/health/topics/tbi/conditioninfo/diagnose. Methods and tests for the diagnosis of TBI are also described, e.g., in Byrnes et al., Front Neuroenergetics, 2013, 5:13; Mutch et al., Neurosurg Clin N Am, 2016, 27:409-439; Prince et al., Brain Sci, 2017, 7:105; and Reis et al., Int J Mol Sci, 2015, 16:11903-11965.

In some embodiments, a subject to be treated (e.g., a subject having a TBI) is identified using one or more cognition and neuropsychological tests. Cognition and neuropsychological tests include, but are not limited to, tests that assess the subject's cognitive (e.g., thinking, reasoning, problem solving, information processing, and memory functions), language, behavioral, motor, and executive functions.

In some embodiments, a subject to be treated (e.g., a subject having a TBI or a subjecting neuroinflammation and/or Tau deposition) is identified using one or more imaging tests. Imaging tests include, but are not limited to, computed tomography (CT), magnetic resonance imaging (MRI), magnetic resonance spectroscopy (MRS), intracranial pressure (ICP) monitoring, or positron emission tomography (PET) imaging. In some embodiments, a subject to be treated is identified by MRI or PET imaging. In some embodiments, PET imaging using a translocator protein radiolabeled ligand (e.g., [¹⁸F]-DPA-714) is used. In some embodiments, a subject to be treated is identified by [¹⁸F]-DPA PET imaging, e.g., as described in the Examples section below.

ACTH Formulations, Dosing, and Administration

In some embodiments, a natural or synthetic ACTH as described herein (e.g., cosyntropin) is administered as a pharmaceutical composition. A pharmaceutical composition may further comprise one or more additional pharmaceutically acceptable components. See, Remington: The Science and Practice of Pharmacy, 21st Ed., 2005, University of the Sciences in Philadelphia (USIP). For example, a pharmaceutical composition can also include, depending on the formulation desired, pharmaceutically-acceptable, non-toxic carriers, excipients, or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

In some embodiments, the method further comprises administering one or more additional pharmaceutical composition further comprises one or more additional therapeutic agents, such as anti-inflammatory agents. In some embodiments, the anti-inflammatory agents are formulated in a pharmaceutical composition, e.g., in a pharmaceutical composition comprising ACTH. Examples of anti-inflammatory agents include, but are not limited to, clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenofibrate, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, and pimecorlimus.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The dosage level of the ACTH depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. In some embodiments, the therapeutically effective dose or efficacious dose of the agent is about 0.001 mg/kg to about 1000 mg/kg daily. For example, a daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. In some embodiments, a pharmaceutical composition may include a dosage of an adrenocorticotropic hormone ranging from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 1 to about 30 mg/kg. The dosage may be adapted by the physician in accordance with conventional factors such as the extent of the disease and different parameters of the subject.

In determining a therapeutically or prophylactically effective dose, in some embodiments, a low dose can be administered and then incrementally increased until a desired response is achieved with minimal or no undesired side effects. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Administration can be achieved in various ways, including oral, buccal, parenteral, intravenous, intradermal, subcutaneous, intramuscular, transdermal, transmucosal, intranasal, intraventricular, etc., administration.

A pharmaceutical composition containing an adrenocorticotropic hormone may be administered in a manner compatible with the dosage formulation and in such amount as is therapeutically effective to result in an improvement or remediation of the symptoms. The pharmaceutical compositions can be in the form of any of a variety of dosage forms, e.g., intravenous dosage forms, subcutaneous dosage forms, and oral dosage forms (e.g., ingestible solutions, drug release capsules). The pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice. Pharmaceutical compositions may be administered to a subject in need thereof, for example, one or more times (e.g., 1-10 times or more) daily, weekly, monthly, biannually, annually, or as medically necessary. Dosages may be provided in either a single or multiple dosage regimens. The timing between administrations may decrease as the medical condition improves or increase as the health of the patient declines.

Duration of Treatment and Treatment Endpoints

In some embodiments, the ACTH (e.g., a full-length ACTH polypeptide or a synthetic derivative of a full-length ACTH polypeptide, e.g., cosyntropin) is administered for a predetermined time, an indefinite time, or until an endpoint is reached. Treatment may be continued on a continuous daily or weekly basis for at least two to three months, six months, one year, or longer. In some embodiments, therapy is for at least 30 days, at least 60 days, at least 90 days, at least 120 days, at least 150 days, or at least 180 days. In some embodiments, treatment is continued for at least 6 months, at least 7 months, at least 8 months, at least 9 months, at least 10 months, at least 11 months, or at least one year. In some embodiments, treatment is continued for the rest of the patient's life or until administration is no longer effective to provide meaningful therapeutic benefit.

In some embodiments, treatment with ACTH results in a reduction in neuroinflammation. In some embodiments, reduction in neuroinflammation is measured by imaging analysis (e.g., by MRI or PET imaging), and/or by biomarker analysis of glial activation, cytokine and chemokine concentration, and/or immune cell activity.

In some embodiments, treatment with ACTH increases brain glucose metabolism. Glucose metabolism in a subject may be measured using one or more known techniques in the art. For example, in some embodiments, brain glucose metabolism is assessed using [18F]-fluorodeoxyglucose (FDG) PET imaging. [¹⁸F]-FDG can be used for the assessment of glucose metabolism in organs such as the heart, lungs, and the brain. Briefly, [¹⁸F]-FDG is taken up by cells, phosphorylated by hexokinase, and retained by tissues with high metabolic activity, resulting in [¹⁸F]-(FDG) that accumulates in brain tissue in proportion to glucose uptake and phosphorylation and is quantifiable using PET imaging. FDG-PET imaging for assessing brain glucose metabolism is described in the art. See, e.g., Byrnes et al., Front Neuroenergetics, 2013, 5:13.

In some embodiments, treatment with ACTH (e.g., cosyntropin) improves one or more characteristics of microglia, such that the microglia more closely resemble “resting” or “non-activated” microglia, as compared to the microglia prior to the onset of treatment. For example, in some embodiments, treatment with ACTH may decrease microglia activation, increase microglia cell count, increase microglia cell area, decrease microglia cell density, and/or increase microglia cell perimeter. As described in the Examples section below, e.g., in Examples 3 and 4, microglia activation may be determined using immunohistochemistry and/or morphological assessment. Immunohistochemistry may be used to assess cytokine release, which is a response following microglia activation. Cytokines that may be tested include, e.g., IL-1beta, IL-4, IL-6, IL-10, TNF-alpha, and COX-2. Microglia activation may also be assessed through biomarkers that are released following pro-inflammatory (M1) and anti-inflammatory (M2) states. For examples, biomarkers IL-1beta and TNF-alpha are indicative of M1 state and Arg-1 and IL-10 are indicative of M2 state.

Further, morphological assessment of microglia activation may be carried out by immunostaining microglia cells using an antibody, e.g., IBA-1. Immunolabeled microglia can then be processed with a microscopy imaging software, e.g., FracLac for ImageJ, to produce morphological parameters for individual microglia. In some embodiments of the methods described herein, the methods may increase microglia cell count, increase microglia cell area, decrease microglia cell density, and/or increase microglia cell perimeter. Once microglia cells are immunostained by an antibody and imaged, cell morphological parameters, such as cell area, density, and perimeter, may be measured by the number of pixels captured in the microscopy image and calculated using available microscopy software, e.g., FracLac for ImageJ. A combined use of microglia phenotypic markers and morphology may be used to assess microglia activation.

In some embodiments, treatment with ACTH increases AMP activated protein kinase (AMPK) activity. Protein kinase activity may be measured using available techniques in the art, e.g., adenylyl cyclase activation assays, luciferase reporter gene assays, and cyclic AMP accumulation assays. Kinase activity within a biological sample may be measured by incubating the immunoprecipitated kinase with an exogenous substrate in the presence of ATP. Measurement of the phosphorylated substrate by a kinase can be assessed by several reporter systems including colorimetric, radioactive, or fluorometric detection. In a cyclic AMP accumulation assay, e.g., GloSensor™, a biosensor with a cyclic AMP binding domain may be fused with a fluorescence reporter, e.g., luciferase. Upon cyclic AMP binding, conformational changes occur that promote large increases in light output.

In further embodiments, the method of treatment involves assessing the subject's improvement at a defined period of time after the onset of treatment, e.g., using one or more cognition, neuropsychological, or imaging tests as described herein, and adjusting the dose of the ACTH that is administered depending upon the outcome of the assessment.

PET Imaging

In some embodiments, an imaging test, such as PET imaging, is used to assess the subject's responsiveness to treatment with ACTH or a derivative thereof. For example, PET imaging can be used to assess the level of neuroinflammation and/or microglia activation status. Without being bound to a particular theory, it is believed that ACTH receptors MCR3/4 regulate M1/M2 microglia polarization during chronic TBI stages (M1, pro-inflammatory; M2, anti-inflammatory). Translocator protein (TSPO) expression, monitored with the radiotracer [¹⁸F]-DPA-714, has been shown to be increased in an animal model in which pro-inflammatory microglia predominate. Thus, [¹⁸F]-DPA-714 PET imaging may be used to identify TBI inflammatory features and ACTH-mediated signaling. The use of [¹⁸F]-DPA-714 PET neuroimaging to identify novel signatures associated with neuroinflammation and ACTH-mediated signaling during TBI progression is further described in the Examples section below, e.g., in Example 8.

### EXAMPLES

The following examples are offered to illustrate, but not to limit, the claimed invention.

### Example 1

### Adrenocorticotropin for Ameliorating Brain Injury

ACTH is one of a group of melanocortin peptides that also include α, β, and γ melanocyte stimulating hormone (MSH) and are synthesized in the pituitary gland from propiomelan-ocortin. They bind to and activate melanocortin receptors (MCRs) which currently are divided into 5 subtypes (MCR1-5). MCR1 is mainly expressed in melanocytes and plays a role in determining skin pigmentation. It also is expressed in monocytes, neutrophils, and lymphocytes, as well as in CNS microglia and astrocytes. MCR2 is responsible for the steroidogenic actions of ACTH by causing cortisol release from the adrenal gland. MCR3 is expressed in the hypothalamus and limbic system and in immunocompetent cells, where it regulates energy homeostasis and inflammatory responses. MCR4 plays a major role in regulating energy homeostasis and MCR4 gene variants cause monogenic obesity. MCR4 also is the most commonly detected MCR in the CNS and is found in the cortex, thalamus, hypothalamus, brain stem, and spinal cord. The roles of MCR5 are less well understood. It is widely distributed in exocrine glands and is found in lymphocytes, and plays a role in sebum production and in thermoregulation.

It is hypothesized that ACTH may target multiple MCRs that could ameliorate the evolution of brain injury, Because the different MCRs are widespread in the CNS, interface with the different components of the central and peripheral inflammatory response, and could enhance cortisol production and the associated anti-inflammatory effects, ACTH may target multiple pathways of the same injury process, making ACTH an attractive therapeutic candidate. For example, ACTH may have a positive effect on pituitary dysfunction. Many TBI patients develop anterior pituitary dysfunction (growth hormone, thyroid and cortisol deficiency) soon or at later stages after injury. Previous studies in adult TBI patients have documented that ACTH deficiency occurs in 0-19% of individuals (Heather et al., J Clin Endocrinol Metab 97:599, 2012). ACTH stimulation of the MCR2 receptor could effectively treat low-cortisol levels in such patients, which may help to maintain plasma and brain glucose levels as well as reduce neuroinflammation.

ACTH may also induce immunomodulatory effects of MCRs. MCRs are anti-inflammatory at cellular (i.e., directly suppressing immunocytes) and systemic levels (i.e., nervous system and glucocorticoid-mediated immune-system down regulation). MCRs also have a direct effect on microglia activation and the release of proinflammatory agents. For example, melanocortins bound to MCR1s on activated microglia suppress production of various proinflammatory mediators such as TNF-α, IL-6 and nitric oxide (Catania et al., Pharmacol Rev. 56:1, 2004). The immunomodulatory effects of MCRs are also described, for example, in Kettenmann et al., (Physiol Rev. 91:461, 2011) and Catania et al. (Pharmacol Rev. 56:1, 2004).

### Example 2

### Effects of Cosyntropin in Rats

In this study, four groups of adult Sprague Dawley rats were studied: (a) sham—with saline (n=2); (b) sham with cosyntropin (n=2); (c) CCI with saline (n=4); and (d) CCI with cosyntropin (n=4). Cosyntropin was administered subcutaneously for 7 days following injury. Sensorimotor function was serially evaluated. At 7 days, the animals were euthanized and studies done to assess perilesional histology and immunohistochemistry.

Methods

Animals. Adult male Sprague-Dawley rats (350-400 g) from Charles River Breeding Labs (Hollister, Calif.) were used as experimental subjects (n=12). Upon arrival, rats were pair housed and acclimated for 1-2 weeks to our standard temperature and lighting conditions (70-76° F., 30-70% humidity, room lights on 07:00-19:00), with food (Teklad 7904) and tap water available ad libitum. All experimental procedures were approved by the Loma Linda University Institutional Use and Animal Care Committee.

Surgical procedures. Animals were anesthetized with isoflurane (2.5-3% in 100% O₂, 2.0 mL/min flow rate) and placed into a stereotaxic frame with the head positioned in a horizontal plane with respect to the interaural line. During all surgical procedures, body temperature was maintained at 36±1° C. using a water cushion. All surgical procedures were performed under aseptic conditions. In brief, following a midline incision, the skin, fascia, and temporal muscle were reflected. Animals receiving CCI were subjected to a 6-mm diameter craniotomy over the right parietal cortex centered at 3 mm posterior and 3.5 mm lateral to bregma. An electromagnetically driven piston (Impact One, Leica Biosystems) was mounted to the stereotactic frame and angled 12° away from vertical, enabling the flat, circular impactor tip (5-mm diameter) to be perpendicular to the surface of the brain at the site of injury. A moderate CCI injury was induced, using ˜5 m/s velocity and 2.0 mm depth of tissue compression for 250 ms. Sham injury rats underwent similar procedures to control for surgical stress and duration of anesthesia, but did not receive any impact. After the scalp was sutured closed, bupivacaine (0.1-0.14 mg/kg, s.c.) was injected around the incision site, the rats were placed in a heated recovery cage (36.0 to 38.0° C.) until ambulatory, and then returned to their home cages.

Experimental design and treatment. Prior to surgery animals were randomly assigned to one of 4 groups: Sham−vehicle (n=2), Sham-cosyntropin (n=2), CCI-vehicle (n=4), and CCI-cosyntropin (n=4). Rats received subcutaneous (SC) injections 30 minutes after the induction of sham or CCI injury and at every 12 hours after that for 7 days. Vehicle injections were 1 ml of 0.9% saline. Cosyntropin was supplied by West Therapeutic Development (Grayslake, Ill.) and given at 60 U/kg/dose for a total daily dose of 120 U/kg/day.

Blood sampling. Venous blood samples (0.1-0.5 mL) were taken at baseline (prior to CCI) and at 7 days post-injury using a 21 G-23 G needle, butterfly needle or lance+restraining tube. Samples were immediately centrifuged (14,000 RPM for 5 minutes) and the plasma frozen at −70° C. until assayed for glucocorticoid levels.

Euthanasia. At the end of the 7 days all animals were euthanized under deep anesthesia and transcardially perfused with saline and 4% paraformaldehyde. The brain (cerebral hemispheres, brainstem, spinal cord, lungs, and spleen were removed.

Histology and immunohistochemistry. After perfusion, the brains were extracted and stored in 4% PFA for 48 hours before transfer to 30% sucrose for cryoprotection for 48 hours. The brains were frozen in Tissue-Tek O.C.T. and sectioned on a CM 3050S cryostat (Leica). Slides were stored at −80° F. until staining. During staining, slides were brought to room temperature and then placed on a slide warmer for 5 minutes. Slides were rinsed in cold 100% ethanol, then washed in 1× phosphate buffered saline (PBS) before being incubated in dilution buffer containing bovine serum albumin, Triton-X, and sodium phosphate buffer. Sections were blocked in 5% goat and 5% donkey serum for 30 minutes. The sections were then incubated in 5% goat serum with primary antibody (IBA-1, Wako, 1:500) overnight at 4° C. Sections were then incubated for one hour at 25° C. with biotinylated secondary antibody (Vector Labs, Burlingame, Calif.). After a 30 minutes incubation with VECTASTAIN Elite ABC Kit (Vector Labs, Burlingame, Calif.), reactions were visualized using diaminobenzidine (DAB). Sections are dipped in ethanol to dehydrate, xylene to clean and cover slipped using permount mounting media.

Results

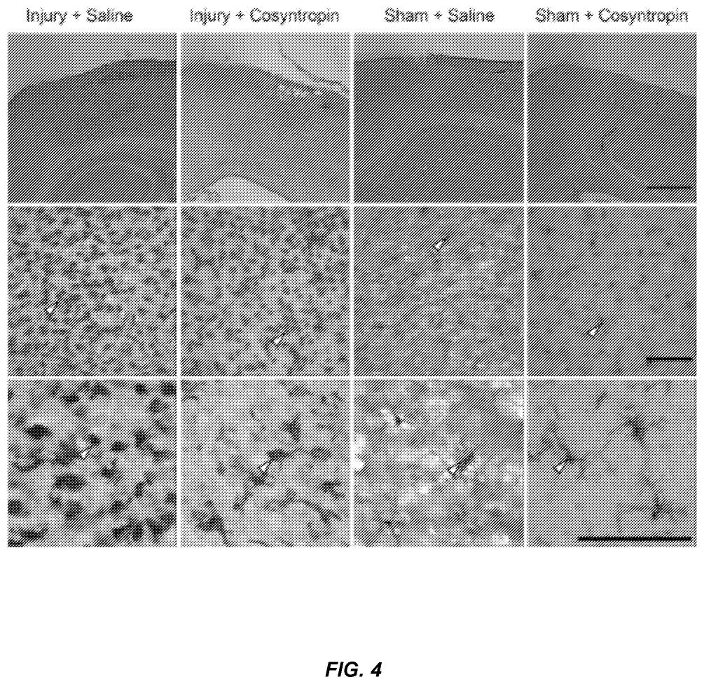

FIG. 4 shows IBA-1 staining for microglia with 4× (upper row), 40× (middle) and 100× (lower) images of CCI injured and sham (craniotomy) rat brains treated with cosyntropin (120 units/kg/day×7 days) or saline. Arrows indicate microglia showing the typical morphology for each treatment. In the injured brain, microglia cells are activated to a greater extent with saline compared to cosyntropin treatment. In CCI-injured animals treated with saline (far left), the lesion areas show significant activation of microglia with thickened and retracted processes suggesting active cytokine release. In contrast, CCI-injured animals treated with cosyntropin show fewer microglia in the lesion area, with a less activated (more branched) morphology. The brains of sham animals show more sparsely distributed resting microglia in the tissue underlying the craniotomy (Scale bar 1 mm at 4× and 100 μm at 40× and 100×). The example demonstrated that cosyntropin improves sensorimotor function and reduces microglia activation in an adult rodent CCI model. The anti-inflammatory roles and mechanisms of ACTH may limit the functional impairments associated with TBI.

### Example 3

### Role of Cosyntropin (Synthetic ACTH 1-24) in Reducing Microglia Activation in a Rodent TBI Model

Melanocortin receptor (MCR1-5) agonists (e.g., ACTH (e.g., cosyntropin)) that target MCR3/R4 ameliorate central and peripheral neuroinflammatory responses and provide a novel therapeutic approach. Following brain injury, quiescent microglia become activated and exhibit both pro-inflammatory (M1) and anti-inflammatory (M2) responses. Activation state of microglia can be evaluated based on morphological features (Fernández-Arjona et al., Frontiers in Cellular Neuroscience 11:235, 2017). The role of cosyntropin (synthetic ACTH 1-24) in reducing microglia activation in a rodent TBI model was examined.

Methods

Experimental Model of TBI. Controlled cortical impact (CCI) model of TBI was used to induce injury in Sprague-Dawley rats. Rats were randomized to four experimental groups: 1) Sham with saline injections (N=2), 2) Sham with cosyntropin injections (N=2), 3) CCI injury with saline injections (N=4) and 4) CCI injury with cosyntropin injections (N=4). Injury was induced with the following parameters: Cortical depth 2.0 mm, speed 5 m/s, and dwell time 250 ms.

Cosyntropin. Rats were administered saline or cosyntropin 30 minutes following surgery followed by saline or drug administration every 12 hours for 7 days following initial injection. Cosyntropin (120 U/kg/day) was supplied by West Therapeutic Development (Grayslake, Ill.).

Microglia Count and Morphology. Cryostat sectioned brains (25 μm) were stained with IBA-1 (Wako, 1:500) followed by VECTASTAIN Elite ABC Kit and visualized with diaminobenzidine (DAB). Images were acquired with a BZ-9000 Keyence microscope (Keyence Corp, Elmwood Park, N.J.). Image processing was conducted with FIJI and image parameters measured with FracLac for ImageJ.

Results

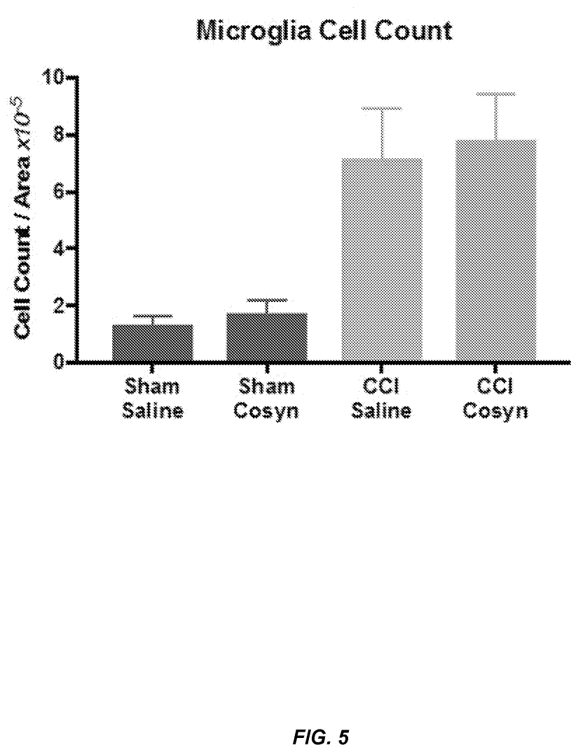

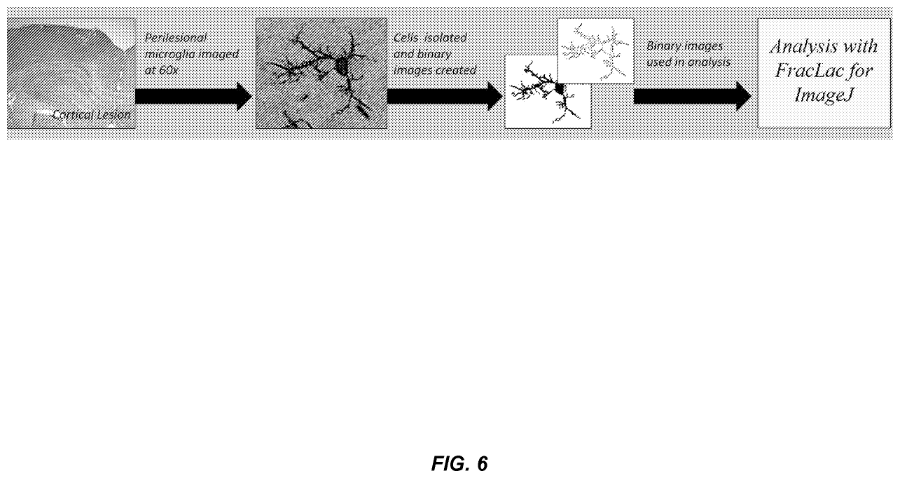

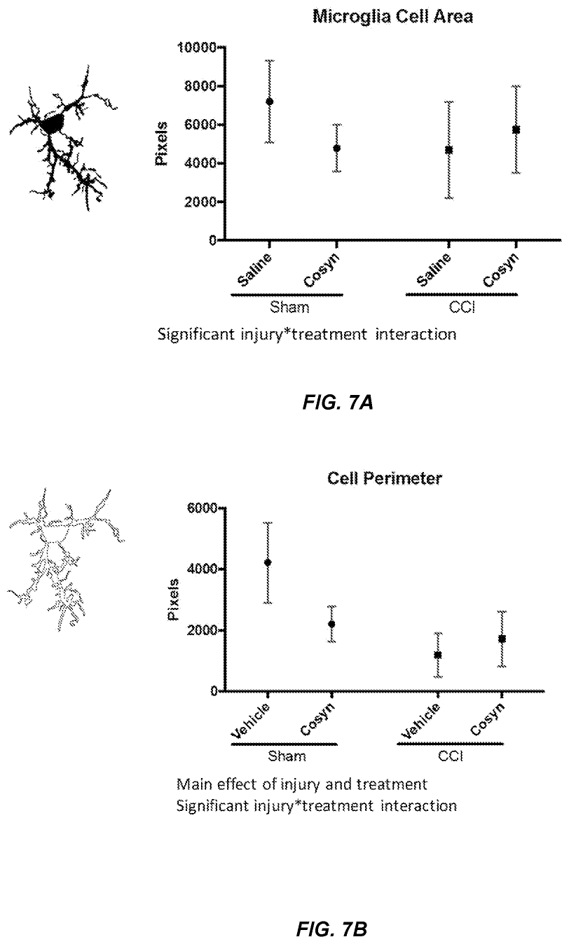

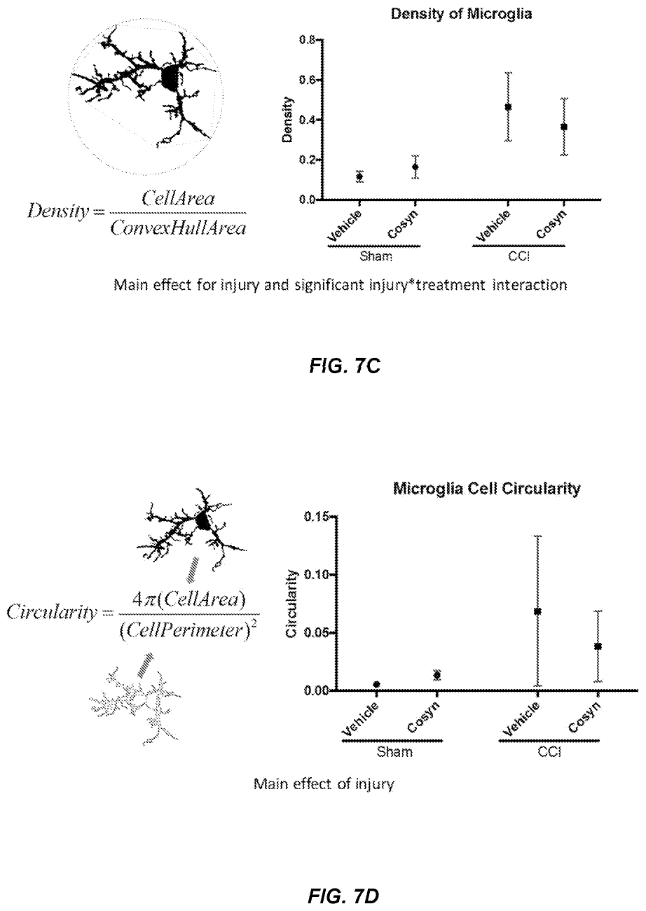

As shown in FIG. 5, unbiased stereological counting methods showed significant difference in number of microglia between sham and CCI treatment in perilesional cortex. No difference was found in the number of microglia between saline and cosyntropin treated groups with either sham or injured groups. Error bars indicate standard deviation. Further, FracLac for ImageJ was used to measure morphological changes in microglia following CCI (Karperien, A., FracLac for ImageJ 1999-2013; available at the ImageJ website) (FIG. 6). CCI altered morphology as demonstrated by decreased perimeter with increased density and circularity. Two-way ANOVA was used to analyze morphological parameters. FIGS. 7A-7D show that cosyntropin reduces CCI-induced morphological changes in microglia.

Conclusion

CCI animals exhibited increased microglia in the lesion site with no difference in cell count between vehicle and treated (FIG. 5). Cosyntropin treatment altered CCI-induced morphological changes as demonstrated by injury/treatment interactions in cell area, cell perimeter, and cell density. Overall, cosyntropin-treated CCI animals showed reduced morphological changes in microglia suggesting a reduced activation state. Decreased activation may decrease the longer term deleterious effects of post-TBI neuroinflammation and may be mediated through the effects of cosyntropin on melanocortin receptors (MCR3/R4) that modulate neuroinflammation.

### Example 4

### Role of Cosyntropin (Synthetic ACTH 1-24) in Ameliorating Inflammation, Lesion Size, and Neurocognitive Deficits Following Experimental TBI in Mice

In studies by other investigators, adult 3-month-old C57BL/6J male mice (Jackson Laboratory, Bar Harbor, Me.) were randomly assigned to TBI or sham groups. In vivo TBI was induced through the controlled cortical impact (CCI) model as previously described (Romine et al., 2014). The location of injury resulted in damage to the primary motor cortex, the medial and lateral parietal association cortex, and septal pole of the hippocampus (George Paxinos and Franklin, 2012). Sham groups experienced the entire procedure except the final impact. A sensitivity power analysis (two-way measure ANOVA: α=0.05, Power=0.8) showed that 23 mice per group was sufficient to reveal minimum effect sizes of 0.30 (G*Power 3.1.9.2, freely available from the University of Düsseldorf).

In our current experiments, cosyntropin was given subcutaneously at a dose of 120 U/kg body weight. In future experiments, cosyntropin (West Therapeutic Development, LLC, Grayslake, Ill.) is administered either subcutaneously or through an intraventricular cannula attached to a subdural osmotic pump (Alzet Durect Corp, Cupertino, Calif., USA). In order to account for brain movement due to edema, a flexible infusion cannula made of plastic tubing is inserted into the lateral ventricle (Guarnieri et al., 2008). Osmotic pump and cannula are placed following the induction of the TBI and administer 0.5 μL of cosyntropin every hour for 7 days.

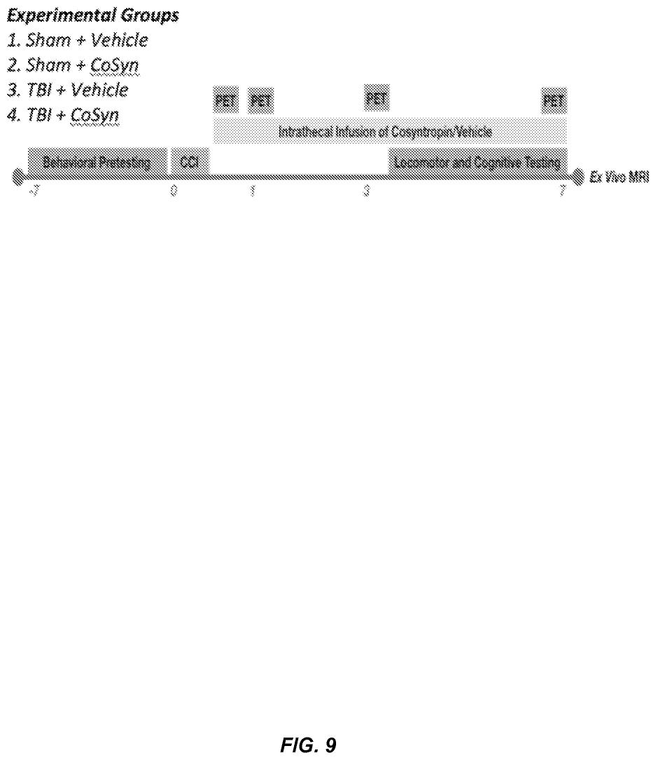

To determine the protective effect of cosyntropin following experimental TBI, adult mice undergo pre- and post-injury behavioral assessment (FIG. 9). Motor function is assessed through commonly used behavioral tests, including Rotarod (RR) and Catwalk (Chen et al., 2014; Shiotsuki et al., 2010). The Morris Water Maze (MWM), open field and novel object recognition (NOR) tests are used to evaluate neurocognitive functioning. MWM pre-training occurs prior to injury followed by assessment at 7 DPI (days post injury). Open field and NOR testing occurs on DPI 4-7 (FIG. 9). Overall protective effect of cosyntropin on brain tissue is assessed via ex vivo magnetic resonance T2-weighted imaging. Ex vivo imaging will allow for the evaluation of lesion volume and edema at 7 DPI.

Modulation of the inflammatory response is evaluated via immunohistochemistry of cytokine expression and microglia activation. Processed and cryostat sectioned brain tissue is stained for cytokine expression (IL-1beta, IL-4, IL-6, IL-10, TNF-alpha, and COX-2) and expression is quantified in both perilesional cortex and hippocampus. Neuroinflammation following TBI is modulated by microglia which exhibit both pro-inflammatory (M1) and anti-inflammatory (M2) states (Donat et al., 2017). Microglia activation is assessed through specific markers for M1 (IL-1beta, TNF-alpha) and M2 (Arg-1, IL-10) phenotypic states and morphological assessment (Donat et al., 2017; Fernández-Arjona et al., 2017). To quantify morphology of microglia, IBA-1 immunolabeled microglia is processed with FracLac for ImageJ to produce morphological parameters (e.g., fractal dimension, lacunarity, density, perimeter) for individual microglia as described previously (Karperien, A., FracLac for ImageJ 1999-2013; available at the ImageJ website, National Institutes of Health; Karperien et al., 2013) (Fernández-Arjona et al., 2017). Analysis of the relationship between microglia phenotypic markers and morphology as well as cell-typing cluster analysis are conducted to support morphological quantification and analysis as a suitable method for identifying and quantifying changes in subpopulations of microglia following experimental TBI (Schweitzer and Renehan, 1997).

### Example 5

### Evaluate the Effect of Cosyntropin on Glucose Metabolism and Expression of MCR4 and MCR4 Downstream Targets Following Experimental TBI in Mice

A similar experimental protocol as described in Example 4 may be followed to assess alterations in expression of MCR4 and MCR4 downstream targets, AMPK and NFkB, following cosyntropin treatment in sham and TBI mice. MCR4 expression is quantified using immunofluorescence to colocalize MCR4 with neurons (NeuN), glia (GFAP), and microglia (IBA-1). Total AMPK and phospho-AMPK levels are measured through Western blot as previously described (Hill et al., 2016). Western blots are also conducted on nuclear and cytosolic NFkB to evaluate the effect of cosyntropin on nuclear translocation of NFkB.

Cosyntropin-mediated alterations in glucose metabolism following experimental TBI is also assessed through the use of [¹⁸F]-fluorodeoxyglucose (FDG) microPET at 30 minutes post injury and DPI 1, 3, and 7 (Qin et al., 2017). Briefly, ¹⁸F-Fluoride is produced on the Siemens Eclipse cyclotron at the LLU Center for Imaging Research and synthesis is carried out in our Radiochemistry Lab using an Explora RN synthesis module. ASIPro Image Analysis software is used to determine ¹⁸F-FDG uptake in the lesioned hemisphere and matching ROI in the contralateral region and cerebellum for pseudo reference. Using ¹⁸F-FDG uptake values, a lesion-to-cerebellum ratio and Standard Uptake Values (SUV) are calculated for each ROI and compared between sham and TBI groups.

### Example 6

### Clinical Study of Neuroinflammation in Former NFL Players

This example describes approaches to the evaluation and management of subjects having symptoms of chronic post-concussion (chronic post-concussion symptoms, PCS) and chronic traumatic encephalopathy (CTE). The disclosure describes detection of neuroinflammation using Positron Emission Tomography (PET) imaging after a traumatic brain injury (TBI) and prognosis about whether treatment of symptomatic subjects (e.g., NFL athletes) with cosyntropin (a synthetic ACTH) would reduce neuroinflammation and improve neuropsychological and neuroimaging outcomes.

Individuals with radiographic evidence of neuroinflammation are randomized into three treatment groups (described herein) that include: (1) standardized rehabilitation; (2) a tailored rehabilitation program that incorporates life-style changes; or (3) pharmacologic treatment with synthetic adrenocorticopin hormone (cosyntropin). Brain imaging is used to study brain injury in, e.g., former NFL players, and when identified, a clinical treatment trial to try to reduce brain inflammation may be proposed. First, medical evaluations and pencil/paper screening to determine if a player has evidence of impaired brain function may be performed. Medical testing is used to see if there is a common condition that is treatable (sleep disorder, hypertension, obesity, diabetes, etc.) and if a problem is detected, the player will be referred for treatment. If screening is positive, a comprehensive battery of psychological tests is performed to better understand what difficulties the player has and at the same time, three kinds of imaging techniques to examine the brain are used. These include (1) positron emission tomography (PET) scanning to look for brain inflammation; (2) Tau PET imaging to look for evidence of CTE; and (3) magnetic resonance imaging and spectroscopy to identify changes in the brain's structure and metabolism.

Evaluation of Subjects using PET Imaging in Combination with Structural MRI/MRS

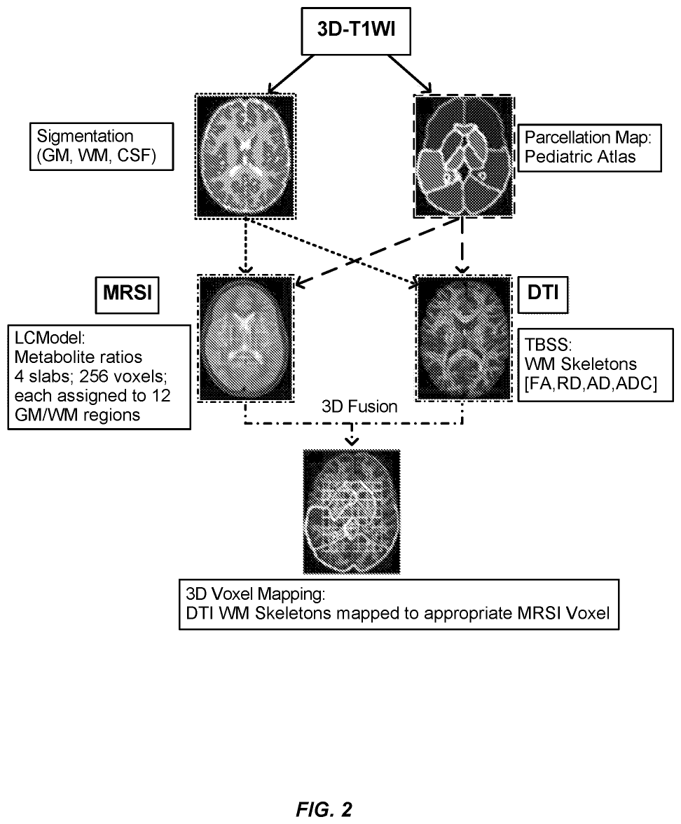

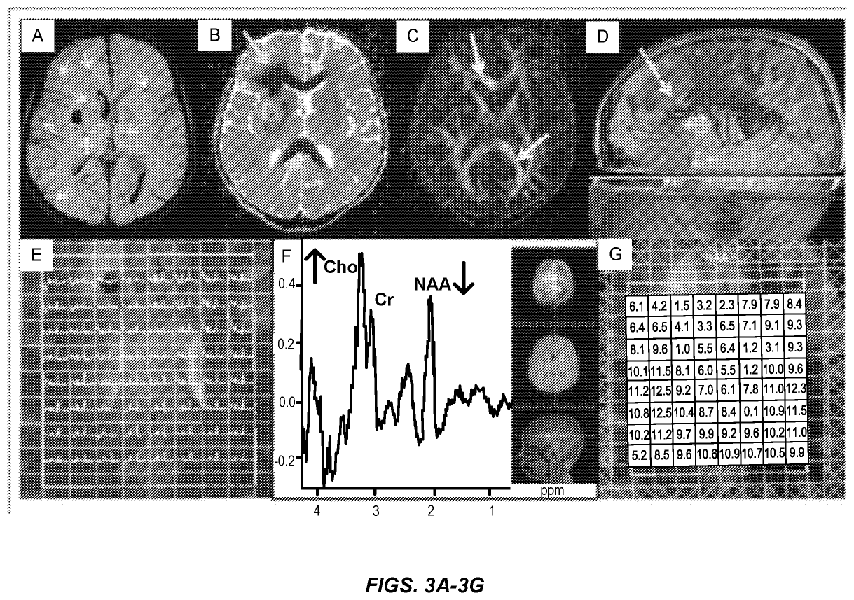

PET imaging in combination with structural MRI/MRS may be used to evaluate affected or symptomatic subjects (e.g., former NFL players), e.g., subjects with persistent (≥6 months) cognitive or behavioral symptoms and objective neuropsychological test abnormalities have evidence of neuroinflammation and/or Tau deposition. An initial evaluation is important as the incidence and severity of neuroinflammation and Tau deposition, as well as structural MRI findings, is unknown in symptomatic subjects (e.g., former NFL players). FIG. 2 shows an exemplary multiparametric MRI/MRS post-processing pipeline used to generate quantitative regional measures of brain diffusivity and metabolite ratios. High resolution T1 weighted MR images are input into an atlas based brain parcellation algorithm and segmented into WM, GM, and CSF tissue classes and 17 different anatomic regions. DTI and MRS data are coaligned with the T1 (or T2) data to transfer diffusion and metabolite information to the T1 space. Finally, diffusivity measures from TBSS and metabolite ratios from individual MRSI voxels are fused to individual brain anatomy and tissue regions to generate tissue content, diffusivity and metabolite measures for each anatomical region. FIGS. 3A-3G show that advanced magnetic resonance imaging (MRI) methods are able to elucidate injuries in different ways and locations. FIGS. 3A-3G analyze the brain injury in a 12-year-old boy, severely injured in a dirt-bike accident at 40 mph, with an initial Glasgow Coma Scale score of 5 (Ashwal et al., J Child Neurol. 29:1704, 2014). MRI was performed 8 days after injury, on a 3.0-Tesla scanner. Susceptibility-weighted imaging (FIG. 3A) shows numerous tiny hemorrhages throughout the brain (small white arrows), many of which were not visible on computed tomography (CT) or MRI. At the same level of the brain, the apparent diffusion coefficient map from diffusion-weighted imaging (FIG. 3B) shows severely restricted water diffusion, suggesting “cytotoxic” changes from cell death in the corpus callosum and right frontal white matter, probably from severe shearing injury. Corresponding color fractional anisotropy map (FIG. 3C) shows accompanying loss of normally symmetric transverse directionality (solid white arrows) of water molecular movement across the corpus callosum (normally red across the genu and splenium). Diffusion tensor imaging tractography (FIG. 3D) depicts the loss of diffusion in the right frontal white matter (dashed white arrow), suggesting impairment or “disruption” of fiber tracts. Multivoxel 3-dimensional magnetic resonance spectroscopy (FIG. 3E) provides information regarding regional metabolite changes and is able to demonstrate additional areas of injury, as highlighted in one (FIG. 3F) of many abnormal voxels within the 3-dimensional volume of tissue studied. Magnetic resonance spectroscopy data also can be displayed using helpful color maps (FIG. 3G) based on the range of metabolite values or ratios, as shown in this color N-acetylaspartate (NAA) map, where the lowest values are colored blue and highest values are colored red.

Clinical Study Design

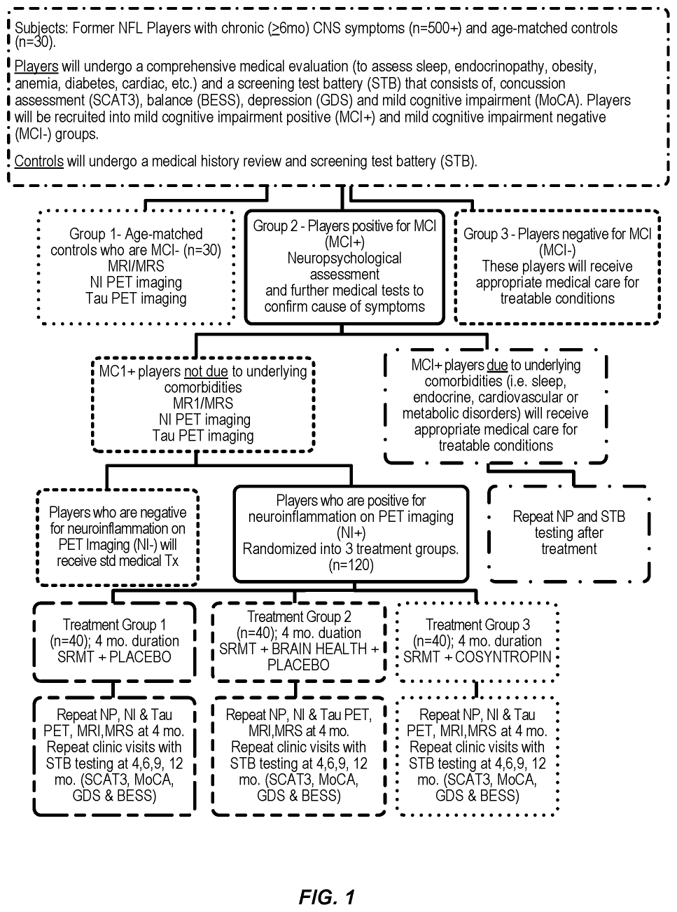

FIG. 1 outlines a clinical study design for identifying and treating symptomatic subjects. In this study, the subjects are former NFL players with chronic (≥6 months) CNS symptoms and age-matched controls. Players will undergo a comprehensive medical evaluation (to assess sleep, endocrinopathy, epilepsy, obesity, vision/hearing impairments, and laboratory screening for common medical conditions (anemia, diabetes, cardiac, etc.) and a screening test battery (STB) that consists of the Sports Concussion Assessment Tool (SCAT3), Montreal Cognitive Assessment tool (MoCA), Geriatric Depression Scale (GDS), and Balance Error Scoring System BESS). Players will be recruited into mild cognitive impairment positive (MCI+) and mild cognitive impairment negative (MCI−) groups. Controls will undergo a medical history review and screening test battery.

MCI+ subjects may undergo further medical evaluation if needed to confirm whether or not there is an underlying co-existent condition to explain their symptoms. For MCI+ subjects with no underlying comorbidity and for age-matched controls who are MCI−, comprehensive neuropsychological testing, neuroinflammation and Tau PET imaging, and MRI/MRS imaging will be performed. Players who are positive for neuroinflammation on PET imagine (NI+) will be randomized to one of 3 treatment groups: (1) Standard Rehabilitation Medical Treatment (SRMT)+placebo; (2) SRMT+rehabilitation program+placebo; and (3) SRMT+cosyntropin. Players who are negative for neuroinflammation on PET imagine (NI−) will receive standard medical treatment.

The mean quantitative imaging variables (QIVs) from neuroinflammation PET, Tau PET, MRI, and MRS between MCI+ and control groups may be compared. Further, the correlation between PET neuroinflammation and Tau QIV may be assessed with neuropsychological testing and with structural/metabolic MRI/MRS variables. An independent t-test is used to compare mean QIV from neuroinflammation PET, Tau PET, MRI, and MRS between MCI+ and control groups. Pearson's correlations are conducted to examine the relationship between PET neuroinflammation and Tau QIV with neuropsychological testing and with structural or metabolic MRI/MRS variables.

### Example 7

### Efficacy of Cosyntropin to Ameliorate Neuroinflammation, Neurodegeneration, and Cognitive Deficits

This experiment uses four groups of anesthetized rats (1. sham+vehicle; 2. sham+CoSyn; 3. TBI+vehicle; TBI+CoSyn). Intracerebroventricular (ICV) cannulation takes place during the CCI surgeries. A sensitivity power analysis (two-way repeated measure ANOVA: (α=0.05 and β=0.2) shows that 12 rats per group is sufficient to reveal minimum effect sizes of 0.30 (G*Power 3.1.9.2). Female and male rats receive continuous ICV cosyntropin infusions (10 μg/d for 7 days) or vehicle. For delayed treatment experiments, cosyntropin administration will commence at 21 days post-injury.

The effects of cosyntropin on gait and locomotor function are evaluated weekly starting at 21 days post-injury (dpi) until 84 dpi using an automated gait analysis system. The rats are imaged using [¹⁸F]-DPA-714 PET at 21 and 84 dpi after CT imaging to facilitate anatomical localization. Briefly, ¹⁸F-Fluoride is produced on a Siemens Eclipse cyclotron at the LLU Center for Imaging Research and synthesis is carried out in the Radiochemistry Lab using an Explora RN synthesis module. [¹⁸F]-DPA-714 PET scans are performed in anesthetized rats using a microPET Rodent R4. Tail vein injections of [¹⁸F]-DPA-714 (25 MBq) are followed by a 20 min list mode acquisition (started 60 min after injection) of a 3D dataset sorted with Fourier rebinning (FORE) to a 2D dataset and reconstructed with the OSEM2D reconstruction algorithm. ASIPro Image Analysis software are used to determine ¹⁸F-DPA uptake in the lesioned hemisphere (co-registration templates are used for sham animals), a matching ROI in the contralateral region, and in the cerebellum are used as a pseudo reference region previously validated for ¹⁸F-DPA-714. Using ¹⁸F-DPA uptake values, a lesion-to-cerebellum ratio (L/C ratio) and Standard Uptake Values (SUV) are calculated for each ROI and compared between sham and TBI groups. The rats are evaluated in a battery of neurobehavioral test from 77 to 84 dpi. The brains are collected for ex vivo MRI (lesion volume and tissue alterations) and immunohistochemical analyses (glial cell response, pro-/anti-inflammatory cytokines, and MCR3/4 activation/expression). The approach reveals the relative contribution of cosyntropin to modulate functional recovery while clarifying its optimal therapeutic windows.

### Example 8

### Identification of [¹⁸F]-DPA-714 PET Neuroimaging Signatures

This experiment aims to identify novel [¹⁸F]-DPA-714 PET neuroimaging signatures associated with neuroinflammation and ACTH-mediated signaling during TBI progression. A combinatorial pharmacological approach using cosyntropin and the selective MCR3/4 antagonist (TBI+vehicle+SHU9119; TBI+cosyntropin+SHU9119) may be used. The timeline and behavioral and imaging protocols described above (e.g., in Example 7) may be used. This experiment reveals that cosyntropin mediates its anti-inflammatory and neuroprotective effects through MCR3/4 mainly by shifting the microglia polarization following TBI. These pathophysiological changes may be monitored using [¹⁸F]-DPA-714 PET, which may be confirmed with immunohistochemical analyses.

### REFERENCES

Abou El Fadl, M. H., and O'Phelan, K. H. (2018). Management of Traumatic Brain Injury. Neurosurgery Clinics of North America 29, 213-221.
Bitto, A., Polito, F., Irrera, N., Calò, M., Spaccapelo, L., Marini, H. R., Giuliani, D., Ottani, A., Rinaldi, M., Minutoli, L., et al. (2012). Protective effects of melanocortins on short-term changes in a rat model of traumatic brain injury*. Critical Care Medicine 40, 945-951.
Catania, A. (2004). Targeting Melanocortin Receptors as a Novel Strategy to Control Inflammation. Pharmacological Reviews 56, 1-29.
Catania, A. (2008). Neuroprotective actions of melanocortins: a therapeutic opportunity. Trends in Neurosciences 31, 353-360.
Chen, S., Zhao, L., Sherchan, P., Ding, Y., Yu, J., Nowrangi, D., Tang, J., Xia, Y., and Zhang, J. H. (2018). Activation of melanocortin receptor 4 with RO27-3225 attenuates neuroinflammation through AMPK/JNK/p38 MAPK pathway after intracerebral hemorrhage in mice. Journal of Neuroinflammation 15.
Chen, Y.-J., Cheng, F.-C., Sheu, M.-L., Su, H.-L., Chen, C.-J., Sheehan, J., and Pan, H.-C. (2014). Detection of subtle neurological alterations by the Catwalk XT gait analysis system. Journal of NeuroEngineering and Rehabilitation 11, 62.
Donat, C. K., Scott, G., Gentleman, S. M., and Sastre, M. (2017). Microglial Activation in Traumatic Brain Injury. Frontiers in Aging Neuroscience 9.
Faden, A. I., and Loane, D. J. (2015). Chronic Neurodegeneration After Traumatic Brain Injury: Alzheimer Disease, Chronic Traumatic Encephalopathy, or Persistent Neuroinflammation? Neurotherapeutics 12, 143-150.
Fernández-Arjona, M. del M., Grondona, J. M., Granados-Durán, P., Fernández-Llebrez, P., and López-Ávalos, M. D. (2017). Microglia Morphological Categorization in a Rat Model of Neuroinflammation by Hierarchical Cluster and Principal Components Analysis. Frontiers in Cellular Neuroscience 11.
George Paxinos, and Franklin, K. (2012). Paxinos and Franklin's the Mouse Brain in Stereotaxic Coordinates (Academic Press).
Giuliani, D., Galantucci, M., Neri, L., Canalini, F., Calevro, A., Bitto, A., Ottani, A., Vandini, E., Sena, P., Sandrini, M., et al. (2014). Melanocortins protect against brain damage and counteract cognitive decline in a transgenic mouse model of moderate Alzheimer's disease. European Journal of Pharmacology 740, 144-150.
Hardie, D. G., Ross, F. A., and Hawley, S. A. (2012). AMPK: a nutrient and energy sensor that maintains energy homeostasis. Nature Reviews Molecular Cell Biology 13, 251-262.
Hill, J. L., Kobori, N., Zhao, J., Rozas, N. S., Hylin, M. J., Moore, A. N., and Dash, P. K. (2016). Traumatic brain injury decreases AMP-activated protein kinase activity and pharmacological enhancement of its activity improves cognitive outcome. Journal of Neurochemistry 139, 106-119.
Ichiyama, T., Sakai, T., Catania, A., Barsh, G. S., Furukawa, S., and Lipton, J. M. (1999). Systemically administered a-melanocyte-stimulating peptides inhibit NF-kB activation in experimental brain inflammation. Brain Res. 836, 31-37.
Kishi, T., Aschkenasi, C. J., Lee, C. E., Mountjoy, K. G., Saper, C. B., and Elmquist, J. K. (2003). Expression of melanocortin 4 receptor mRNA in the central nervous system of the rat. The Journal of Comparative Neurology 457, 213-235.
Lisak, R. P., Nedelkoska, L., Bealmear, B., and Benjamins, J. A. (2015). Melanocortin receptor agonist ACTH 1-39 protects rat forebrain neurons from apoptotic, excitotoxic and inflammation-related damage. Experimental Neurology 273, 161-167.
Morgan, D. A., McDaniel, L. N., Yin, T., Khan, M., Jiang, J., Acevedo, M. R., Walsh, S. A., Ponto, L. L. B., Norris, A. W., Lutter, M., et al. (2015). Regulation of Glucose Tolerance and Sympathetic Activity by MC4R Signaling in the Lateral Hypothalamus. Diabetes 64, 1976-1987.
Qin, X., You, H., Cao, F., Wu, Y., Peng, J., Pang, J., Xu, H., Chen, Y., Chen, L., Vitek, M. P., et al. (2017). Apolipoprotein E Mimetic Peptide Increases Cerebral Glucose Uptake by Reducing Blood-Brain Barrier Disruption after Controlled Cortical Impact in Mice: An 18 F-Fluorodeoxyglucose PET/CT Study. Journal of Neurotrauma 34, 943-951.
Rabinovitch, R. C., Samborska, B., Faubert, B., Ma, E. H., Gravel, S.-P., Andrzejewski, S., Raissi, T. C., Pause, A., St.-Pierre, J., and Jones, R. G. (2017). AMPK Maintains Cellular Metabolic Homeostasis through Regulation of Mitochondrial Reactive Oxygen Species. Cell Reports 21, 1-9.
Schweitzer, L., and Renehan, W. (1997). The use of cluster analysis for cell typing. Brain Research Protocols 1, 100-108.
Shiotsuki, H., Yoshimi, K., Shimo, Y., Funayama, M., Takamatsu, Y., Ikeda, K., Takahashi, R., Kitazawa, S., and Hattori, N. (2010). A rotarod test for evaluation of motor skill learning. Journal of Neuroscience Methods 189, 180-185.
Simon, D. W., McGeachy, M. J., Bayir, H., Clark, R. S. B., Loane, D. J., and Kochanek, P. M. (2017). The far-reaching scope of neuroinflammation after traumatic brain injury. Nature Reviews Neurology 13, 171-191.
Wang, Y., Huang, Y., Xu, Y., Ruan, W., Wang, H., Zhang, Y., Saavedra, J. M., Zhang, L., Huang, Z., and Pang, T. (2018). A Dual AMPK/Nrf2 Activator Reduces Brain Inflammation After Stroke by Enhancing Microglia M2 Polarization. Antioxidants & Redox Signaling 28, 141-163.
Xiong, Y., Mahmood, A., and Chopp, M. (2018). Current understanding of neuroinflammation after traumatic brain injury and cell-based therapeutic opportunities. Chinese Journal of Traumatology 21, 137-151.

One or more features from any embodiments described herein or in the figures may be combined with one or more features of any other embodiment described herein in the figures without departing from the scope of the disclosure.

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed is:

1. A method of treating traumatic brain injury (TBI), comprising administering to a subject having a TBI a therapeutically effective amount of an adrenocorticotropic hormone (ACTH).

2. The method of claim 1, wherein the ACTH is a full-length ACTH polypeptide.

3. The method of claim 1, wherein the ACTH is a synthetic derivative of a full-length ACTH polypeptide.

4. The method of claim 3, wherein the ACTH is cosyntropin.

5. The method of claim 1, wherein the subject has neuroinflammation and/or Tau deposition in one or more brain regions.

6. The method of claim 5, wherein the neuroinflammation and/or Tau deposition is identified by magnetic resonance imaging (MRI) and/or positron emission tomography (PET) imaging.

7. The method of claim 1, wherein the traumatic brain injury is caused by a fall, an assault, a motor vehicle accident, a sport or recreational injury, shaken baby syndrome, a gunshot wound, a combat injury, or an electric shock.

8. The method of claim 1, wherein the subject has one or more neurological symptoms selected from the group consisting of memory loss, depression, mood swings, balance problems, anger, aggression, anxiety, substance abuse, obsessive compulsive disorder, and muted emotions.

9. A method of reducing neuroinflammation and/or Tau deposition in one or more brain regions in a subject in need thereof, comprising administering to the subject a therapeutically effective amount of an ACTH.

10. The method of claim 9, wherein the neuroinflammation and/or Tau deposition is identified by magnetic resonance imaging (MRI) and/or positron emission tomography (PET) imaging.

11. The method of claim 9, wherein the ACTH is a full-length ACTH polypeptide.

12. The method of claim 9, wherein the ACTH is a synthetic derivative of a full-length ACTH polypeptide.

13. The method of claim 12, wherein the ACTH is cosyntropin.

14. The method of claim 9, wherein the subject has one or more neurological symptoms selected from the group consisting of memory loss, depression, mood swings, balance problems, anger, aggression, anxiety, substance abuse, obsessive compulsive disorder, and muted emotions.

15.-20. (canceled)
